(19) United States
(12) Patent Application Publication
(10) Pub. No.: US 2021/0379316 A1
(43) Pub. Date: Dec. 9, 2021

(54) RESPIRATORY FILTER AND CONDENSATE MANAGEMENT APPARATUS

(71) Applicant: TELEFLEX MEDICAL INCORPORATED, Morrisville, NC (US)

(72) Inventors: Daniel Patrick DWYER, Cary, NC (US); Christopher JACKSON, Raleigh, NC (US); Douglas DREW, Raleigh, NC (US)

(21) Appl. No.: 17/410,576

(22) Filed: Aug. 24, 2021

Related U.S. Application Data
(63) Continuation of application No. PCT/US2020/019671, filed on Feb. 25, 2020.
(60) Provisional application No. 62/810,856, filed on Feb. 26, 2019.

(51) Int. Cl. A61M 16/08; B01D 46/52; B01D 46/00; B01D 46/42; A61M 16/10; A61M 16/20

(52) U.S. Cl. CPC A61M 16/0808; B01D 46/521; B01D 46/0031; A61M 16/208; B01D 46/0032; A61M 16/1065; A61M 16/0891; B01D 46/4272

(57) ABSTRACT

A respiratory filter and condensate management apparatus is provided for use in a breathing circuit during patient respiration. The apparatus includes a housing having an air inlet port for receiving a flow of respiratory air, and an air outlet port for outputting the flow of respiratory air to a ventilator. A filter compartment is provided within the housing and includes a filter member located in a flow path of the respiratory air from the air inlet port to the air outlet port. A condensate collection compartment is provided within the housing and includes at least one reservoir and at least one self-sealing drainage port. The reservoir collects liquid formed by condensation of the respiratory air within the filter compartment, and the drainage port allows removal of the collected liquid from the reservoir while the housing remains connected to the breathing circuit.

### CROSS-REFERENCE TO RELATED APPLICATIONS

This application is a continuation of International Patent Application No. PCT/US2020/019671 filed on Feb. 25, 2020, and published as WO 2020/176481, which claims priority to U.S. Provisional Patent Application No. 62/810,856 filed Feb. 26, 2019. The contents of these applications are incorporated herein by reference in their entirety.

### FIELD OF THE INVENTION

The present disclosure generally relates to respiratory filters for use in a breathing circuit, and more particularly, to respiratory filters operable to manage an accumulation of condensate produced during active humidification.

### BACKGROUND

Ventilators are commonly used to aid or replace a patient's respiratory function. Such ventilators are typically connected to a breathing circuit having an inspiratory limb for delivering a flow of air from the ventilator to the patient for inhalation, and an expiratory limb for returning a flow of exhaled air from the patient back to the ventilator. The flow of air is often heated and humidified to increase patient comfort and compliance during respiration.

A respiratory filter may be used in the breathing circuit between the expiratory limb and the ventilator in order to prevent the ventilator from being contaminated by bacteria, viruses, and/or other waste material exhaled by the patient during respiration. The exhaled air from the patient contains vapor that may form condensate within the expiratory limb. Similarly, vapor that reaches the respiratory filter may form condensate within the filter. Some conventional expiratory limbs may be heated, such as by a heating wire, to reduce the amount of condensate within the expiratory limb. However, conventional respiratory filters are unheated, and are therefore exposed to room air that is almost always colder than the air inside the filter. Thus, unwanted condensation or rainout commonly occurs within the filter when the heated and humidified air enters the colder filter.

The accumulation of condensate within the respiratory filter can saturate the filter material causing it to perform less effectively. For instance, resistance to air flow in the filter material may increase as the filter material becomes more saturated with water. Additionally, condensate that forms in the filter may be contaminated with bacteria, viruses, and/or other waste material from the patient.

Some typical respiratory filters may include a container to collect moisture on the circuit side of the filter. However, such filters routinely fill up with water and require dismantling or breaking of the breathing circuit in order to drain the excess fluid. It is advantageous to avoid taking apart the breathing circuit during respiration in order to maintain a positive end expiratory pressure (PEEP) and minimize the risk of infection. Moreover, ventilation patterns can be affected when conventional respiratory filters fill up with liquid from condensation, which can cause false triggering on the ventilator. Furthermore, known heated filters either require a ventilator that has a heating capability, a standalone filter heater, or a powered humidifier, each of which blocks viewing of the filter and are also cost prohibitive.

Accordingly, there is a clear need for a respiratory filter with integrated liquid drainage for condensate management during active humidification. In particular, the respiratory filter and condensate management apparatus of the present disclosure mitigates the buildup of liquid within the expiratory filter, minimizes filter change-outs, helps maintain PEEP in the breathing circuit, and mitigates the risk of infection due to breaking of the circuit, among other advantages.

### SUMMARY

The foregoing needs are met, to a great extent, by the present disclosure of a respiratory filter and condensate management apparatus discussed herein. The respiratory filter and condensate management apparatus may comprise a housing having an air inlet port and an air outlet port, the air inlet port configured to connect to an expiratory limb of the breathing circuit for receiving a flow of expiratory air, and the air outlet port configured to connect to a ventilator for outputting the flow of expiratory air; a filter compartment provided within the housing, the filter compartment being in communication with the air inlet port and the air outlet port; a filter member provided within the filter compartment and located in a flow path of the expiratory air from the air inlet port to the air outlet port; a condensate collection compartment provided within the housing, the condensate collection compartment being adjacent to the filter compartment and including a first reservoir operable to collect liquid formed by condensation of the expiratory air within the filter compartment; and a first self-sealing drainage port provided in a wall of the condensate collection compartment, the first self-sealing drainage port operable to allow removal of the collected liquid from the first reservoir without disconnecting the filter from the breathing circuit.

According to another aspect of the disclosure, the first reservoir may be configured to collect liquid formed by condensation of the expiratory air within a patient side of the filter compartment before the flow of expiratory air passes through the filter member.

According to another aspect of the disclosure, a first condensate passage is operable to permit the liquid condensation within the filter compartment to pass into the first reservoir of the condensate collection compartment.

According to another aspect of the disclosure, the first self-sealing drainage port comprises a self-closing one-way valve operable to allow suctioning out the condensate collected in the first reservoir.

According to another aspect of the disclosure, the condensate collection compartment further includes a second reservoir operable to collect liquid formed by condensation of the expiratory air within the filter compartment.

According to another aspect of the disclosure, the second reservoir is configured to collect condensation of the expiratory air within a ventilator side of the filter compartment after the flow expiratory air passes through the filter member.

According to another aspect of the disclosure, a second self-sealing drainage port may be provided in a wall of the condensate collection compartment, the second self-sealing drainage port operable to allow removal of the collected liquid from the second reservoir.

According to another aspect of the disclosure, the second self-sealing drainage port comprises a self-closing valve operable to allow suctioning out the condensate collected in the second reservoir.

According to another aspect of the disclosure, a second condensate passage may be operable to permit the liquid condensation within the filter compartment to pass into the second reservoir of the condensate collection compartment.

According to another aspect of the disclosure, the first and second reservoirs in the condensate collection compartment are separated by a partition configured to prevent mixing the liquid collected in the first reservoir with the liquid collected in the second reservoir.

According to another aspect of the disclosure, an impact pad may be provided within the filter compartment and operable to remove liquid from the flow of expiratory air.

According to another aspect of the disclosure, the impact pad may be located in a patient side of the filter compartment between the filter member and the air inlet port.

According to another aspect of the disclosure, the impact pad directly faces the air inlet port in a direction perpendicular to the flow of air.

According to another aspect of the disclosure, the impact pad may be suspended within the filter compartment.

According to another aspect of the disclosure, a clearance surrounding a periphery of the impact pad allows the flow of expiratory air to be diverted around the impact pad toward the filter member.

According to another aspect of the disclosure, the impact pad may be mounted to a mounting frame within the patient side of the filter compartment, the mounting frame including an opening configured to allow the flow of air to pass from the air inlet port to the filter member through the impact pad.

According to another aspect of the disclosure, a surface of the impact pad may be flat.

According to another aspect of the disclosure, the impact pad may be an electrostatic pad.

According to another aspect of the disclosure, the filter member may be pleated.

According to another aspect of the disclosure, a respiratory filter and condensate management apparatus for a breathing circuit may comprise: a housing having an air inlet port and an air outlet port, the air inlet port configured to connect to a breathing circuit for receiving a flow of respiratory air, and the air outlet port configured to connect to a ventilator for outputting the flow of respiratory air; a filter compartment provided within the housing, the filter compartment defining a patient side in communication with the air inlet port and a ventilator side in communication with the air outlet port; a filter member provided within the filter compartment and located in a flow path of the respiratory air from the air inlet port to the air outlet port; a condensate collection compartment provided within the housing and including a reservoir and a self-sealing drainage port, the reservoir configured to collect liquid formed by condensation of the respiratory air within the filter compartment, and the self-sealing drainage port operable to prevent a leakage of the respiratory air for maintaining pressure in the breathing circuit.

According to another aspect of the disclosure, the self-sealing drainage port may comprise a one-way valve operable to allow removal of the collected liquid from the reservoir while the housing is connected to the breathing circuit.

According to another aspect of the disclosure, an impact pad may be provided within the filter compartment and operable to remove liquid from the flow of respiratory air.

According to another aspect of the disclosure, the impact pad may be located in the patient side of the filter compartment between the filter member and the air inlet port.

According to another aspect of the disclosure, the impact pad may be suspended within the filter compartment and may be operable to divert the flow of respiratory air around a periphery of the impact pad and toward the filter member.

According to another aspect of the disclosure, the impact pad may be mounted to a mounting frame within the patient side of the filter compartment, the mounting frame including an opening configured to allow the flow of air to pass from the air inlet port to the filter member through the impact pad.

According to another aspect of the disclosure, an apparatus is provided for removing liquid particles from a flow of respiratory air within a breathing circuit, the apparatus comprising: a housing having an air inlet port and an air outlet port, the air inlet port configured to connect to a breathing circuit for receiving a flow of respiratory air, and the air outlet port configured to connect to a ventilator for outputting the flow of respiratory air; a filter compartment provided within the housing, the filter compartment defining a patient side in communication with the air inlet port and a ventilator side in communication with the air outlet port; a filter member provided within the filter compartment and located in a flow path of the respiratory air from the air inlet port to the air outlet port; and an impact pad within the patient side of the filter compartment and operable to remove liquid particles from the flow of respiratory air.

According to another aspect of the disclosure, the impact pad may be located between the filter member and the air inlet port.

According to another aspect of the disclosure, the impact pad may be suspended within the filter compartment and is operable to divert the flow of respiratory air around a periphery of the impact pad and toward the filter member.

According to another aspect of the disclosure, a clearance surrounding a periphery of the impact pad may be provided that allows the flow of respiratory air to be diverted around the impact pad toward the filter member.

According to another aspect of the disclosure, the impact pad may be mounted to a mounting frame within the patient side of the filter compartment, the mounting frame including an opening configured to allow the flow of air to pass from the air inlet port to the filter member through the impact pad.

According to another aspect of the disclosure, the impact pad may be an electrostatic pad.

According to another aspect of the disclosure, a condensate collection compartment may be provided within the housing and including a reservoir and a self-sealing drainage port.

According to another aspect of the disclosure, the reservoir may be configured to collect liquid formed by condensation of the respiratory air within the filter compartment.

According to another aspect of the disclosure, the self-sealing drainage port may be operable to prevent a leakage of the respiratory air for maintaining pressure in the breathing circuit.

According to another aspect of the disclosure, the self-sealing drainage port may comprise a one-way valve operable to allow removal of the collected liquid from the reservoir while the housing is connected to the breathing circuit.

There has thus been outlined certain embodiments of the disclosure in order that the detailed description thereof herein may be better understood, and in order that the present contribution to the art may be better appreciated. There are additional embodiments of the disclosure that will be described below and which form the subject matter of the claims appended hereto.

In this respect, before explaining at least one aspect of the respiratory filter and condensate management apparatus in detail, it is to be understood that the respiratory filter and condensate management apparatus is not limited in its application to the details of construction and to the arrangements of the components set forth in the following description or illustrated in the drawings. The respiratory filter and condensate management apparatus is capable of aspects in addition to those described, and of being practiced and carried out in various ways. Also, it is to be understood that the phraseology and terminology employed herein, as well as the abstract, are for the purpose of description and should not be regarded as limiting.

As such, those skilled in the art will appreciate that the conception upon which this disclosure is based may readily be utilized as a basis for the designing of other structures, methods, and systems for carrying out the several purposes of the respiratory filter and condensate management apparatus. It is important, therefore, that the claims be regarded as including such equivalent constructions insofar as they do not depart from the spirit and scope of the disclosure.

### BRIEF DESCRIPTION OF THE DRAWINGS

In order that the disclosure may be readily understood, aspects of the respiratory filter and condensate management apparatus are illustrated by way of examples in the accompanying drawings, in which like parts are referred to with like reference numerals throughout.

FIG. 1 is a perspective view of a respiratory filter and condensate management apparatus according to an implementation of the present disclosure.

FIG. 2 is a side elevation view of the respiratory filter and condensate management apparatus of FIG. 1.

FIG. 3 is a cross-sectional side elevation view of the respiratory filter and condensate management apparatus taken along line 3-3 of FIG. 2.

FIG. 4 is a cross-sectional perspective view of the respiratory filter and condensate management apparatus of FIG. 3.

FIG. 5 is a front perspective view of a valve assembly of the respiratory filter and condensate management apparatus in accordance with the present disclosure.

FIG. 6 is a rear perspective view of the valve assembly of FIG. 5.

FIG. 7 is a perspective view of a respiratory filter and condensate management apparatus according to another implementation of the present disclosure.

FIG. 8 is a side elevation view of the respiratory filter and condensate management apparatus of FIG. 7.

FIG. 9 is a cross-sectional view of the respiratory filter and condensate management apparatus taken along line 9-9 of FIG. 8.

FIG. 10 is a cross-sectional perspective view of the respiratory filter and condensate management apparatus of FIG. 9.

FIG. 11 is a perspective view of an apparatus according to another implementation of the present disclosure.

FIG. 12 is a cross-sectional view of the apparatus depicted in FIG. 11.

### DETAILED DESCRIPTION

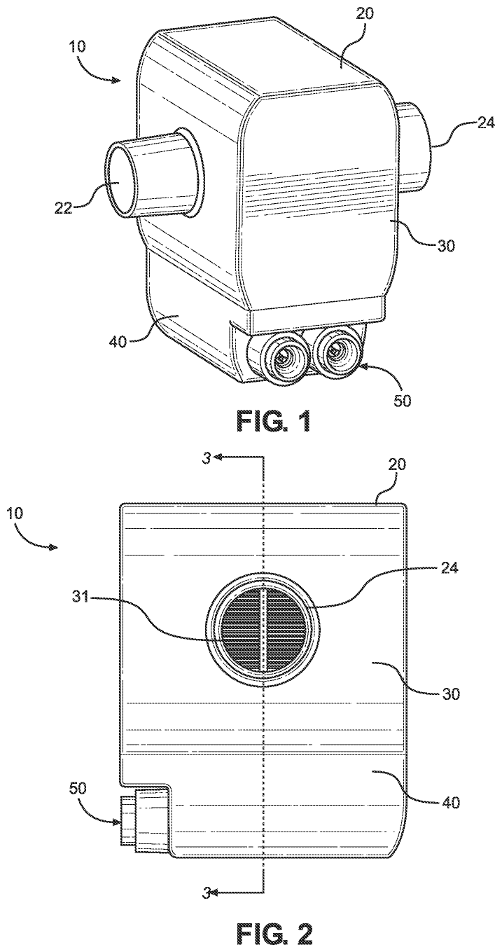

FIGS. 1-4 illustrate a respiratory filter and condensate management apparatus 10 for a breathing circuit in accordance with an implementation of the present disclosure. The respiratory filter and condensate management apparatus 10 comprises a housing 20 having a filter compartment 30 and a condensate collection compartment 40. The filter compartment 30 includes an air inlet port 22 and an air outlet port 24. The air inlet port 22 is configured to connect to an expiratory limb of the breathing circuit, and the air outlet port 24 is configured to connect to a ventilator.

A filter member 31 is provided within the filter compartment 30 and is in communication with the air inlet port 22 and the air outlet port 24 for filtering bacteria, viruses, medicament, and/or other waste material exhaled by a patient during respiration. More particularly, the filter member 31 is located in an air flow path from the air inlet port 22 to the air outlet port 24. The filter member 31 may be made from a material, such as micro-fiberglass, that is operable to catch bacteria, viruses, medicament, and/or other waste material, while still allowing the flow of air to pass through the filter member. For instance, the filter member 31 may be a high efficiency particulate air (HEPA) filter, or an ultra low particulate air (ULPA) filter, among others. In some aspects, the filter member 31 may be pleated to improve efficiency of the air filtration, while in other aspects, the filter member may be non-pleated. The filter member 31 may also be capable of letting through gaseous vapor comprising small water droplets. Further, the filter member may be treated with an antimicrobial agent.

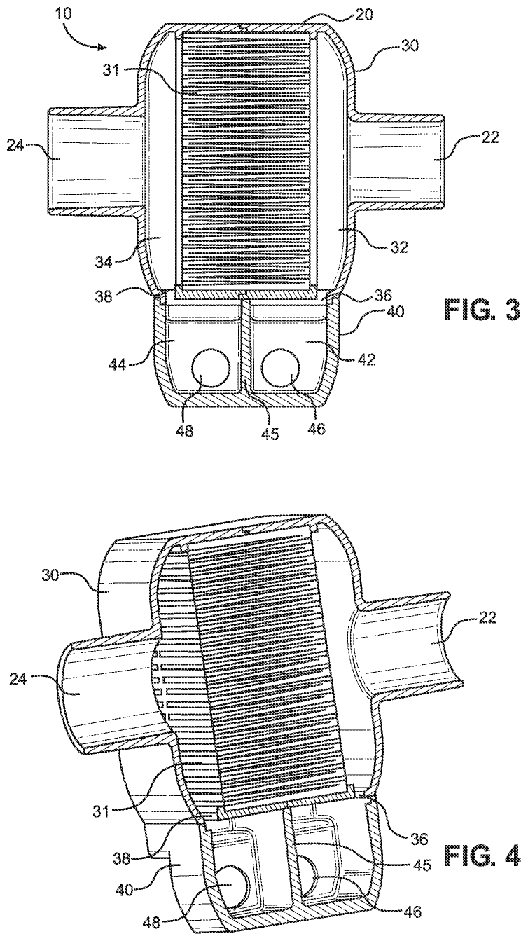

As shown in FIGS. 3 and 4, the filter member 31 is configured to fit within the filter compartment 30 such that the flow of air from the air inlet port 22 to the air outlet port 24 must pass through the filter member 31. Stated another way, the filter member 31 has a shape and size relative to a shape and size of the filter compartment 30 such that the flow of air from the air inlet port 22 to the air outlet port 24 is prevented from bypassing the filter member 31. As illustrated in FIGS. 3 and 4, the filter member 31 may have a generally cuboidal shape, among others.

Heated and humidified air that enters the air inlet port 22 may produce condensate within the filter compartment 30 due to the colder air within the filter compartment. Condensation or rainout may occur in the patient side 32 of the filter compartment 30 before the air flow passes through the filter member 31, and thus condensate that accumulates within the patient side of the filter compartment may be dirty water. As previously described, the filter member 31 may be capable of letting through gaseous vapor comprising water droplets. Accordingly, condensation or rainout may also occur in the ventilator side 34 of the filter compartment 30 after the air flow passes through the filter member 31, and thus condensate that accumulates within the ventilator side of the filter compartment may be clean water.

The condensate collection compartment 40 is located directly adjacent to and below the filter compartment 30, and is configured to collect liquid water formed by condensation within the filter compartment. In particular, the condensate collection compartment 40 includes a first reservoir 42 operable to collect liquid formed by condensation within the patient side 32 of the filter compartment 30, and a second reservoir 44 operable to collect liquid formed by condensation within the ventilator side 32 of the filter compartment. A partition 45 is provided within the condensate collection compartment 40 between the first and second reservoirs 42, 44 to separate the reservoirs and ensure the dirty water within the first reservoir 42 does not mix with the clean water in the second reservoir 44. In some aspects, the filter compartment 30 and the condensate collection compartment 40 may be integrally formed. In other aspects, the condensate collection compartment 40 may be removably attached to the filter compartment 30.

During use in patient respiration, liquid condensation that forms within the filter compartment 30 will drip downward toward the condensate collection compartment 40 due to the effect of gravity. More specifically, condensate within the patient side 32 of the filter compartment 30 will pass downward through a first condensate passage 36 and into the first reservoir 42 of the condensate collection compartment 40 due to gravity. Similarly, condensate within the ventilator side 34 of the filter compartment 30 will pass downward through a second condensate passage 38 and into the second reservoir 44 of the condensate collection compartment 40 due to gravity.

A first drainage port 46 is provided in a wall of the condensate collection compartment 40 and is operable to allow removal of the liquid collected in the first reservoir 42. Similarly, a second drainage port 48 is provided in a wall of the condensate collection compartment 40 and is operable to allow removal of the liquid collected in the second reservoir 44. Each of the first and second drainage ports 46, 48 may be self-sealing to prevent a leakage of air for maintaining pressure in the breathing circuit, and also to prevent a leakage of liquid. More particularly, each of the first and second drainage ports 46, 48 may comprise a respective self-closing valve assembly 50 operable to allow suctioning of the liquid condensation collected in the respective first and second reservoirs 42, 44. Such suctioning may be performed by placing a suction wand, or other suction conduit connected to a vacuum source, into the valve assembly 50.

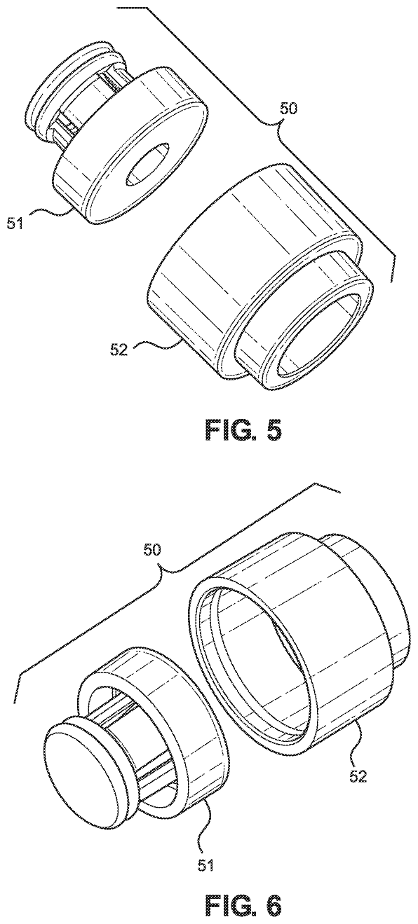

As shown in FIGS. 5 and 6, the valve assembly 50 comprises a one-way check valve 51, such as a ball check valve, and a valve cover 52 placed over the check valve. The one-way check valve 51 is configured to prevent a leakage of air in order to maintain pressure in the breathing circuit. The one-way check valve 51 also allows the liquid to flow out of the reservoir and prevents the liquid from flowing back into the reservoir. Furthermore, as depicted in FIGS. 1 and 2, the first and second drainage ports 46, 48 are both provided in the same side wall of the condensate collection compartment 40 to provide better ergonomic positioning for increased suctioning efficiency. It should be appreciated that according to some aspects, the first and second drainage ports may be provided on different side walls of the condensate collection compartment. Additionally, the condensate collection compartment 40 may include a cut-out portion or stepped region configured to receive a respective valve assembly 50 such that the valve assembly does not extend beyond an outer wall of the housing. Such an arrangement helps prevent or minimize damage to the valve assembly 50 that may be caused by accidentally bumping it or knocking it during use.

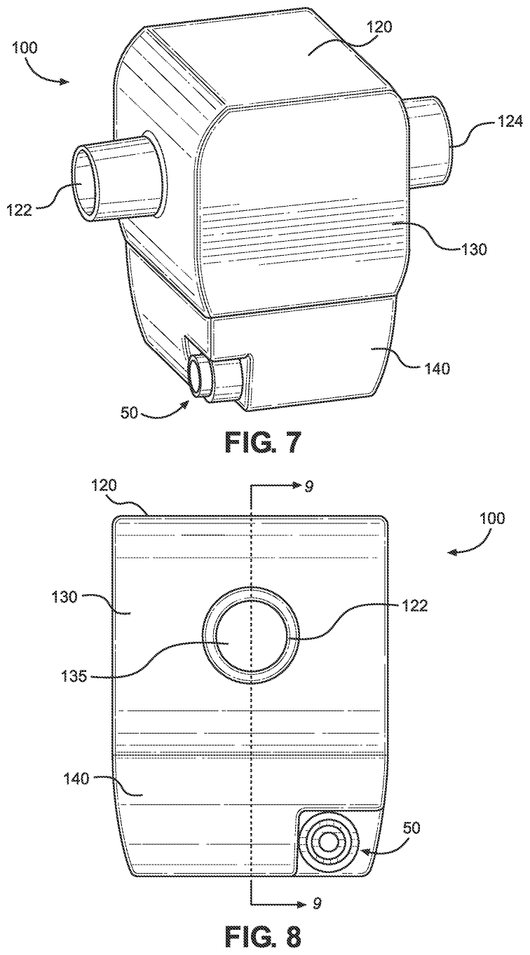

Turning to FIGS. 7-10, a respiratory filter and condensate management apparatus 100 for a breathing circuit is illustrated in accordance with another implementation of the present disclosure. The respiratory filter and condensate management apparatus 100 comprises a housing 120 having a filter compartment 130 and a condensate collection compartment 140. The filter compartment 130 includes an air inlet port 122 and an air outlet port 124. The air inlet port 122 is configured to connect to an expiratory limb of the breathing circuit, and the air outlet port 124 is configured to connect to a ventilator.

A filter member 131 is provided within the filter compartment 130 and is in communication with the air inlet port 122 and the air outlet port 124 for filtering bacteria, viruses, medicament, and/or other waste material exhaled by a patient during respiration. More particularly, the filter member 131 is located in an air flow path from the air inlet port 122 to the air outlet port 124. The filter member 131 may be made from a material, such as micro-fiberglass, that is operable to catch bacteria, viruses, medicament, and/or other waste material, while still allowing the flow of air to pass through the filter member. For instance, the filter member 131 may be a high efficiency particulate air (HEPA) filter, or an ultra low particulate air (ULPA) filter, among others. The filter member 131 may be pleated to improve efficiency of the air filtration. According to some aspects, the filter member 131 may also be capable of letting through gaseous vapor comprising small water droplets. Further, the filter member may be treated with an antimicrobial agent.

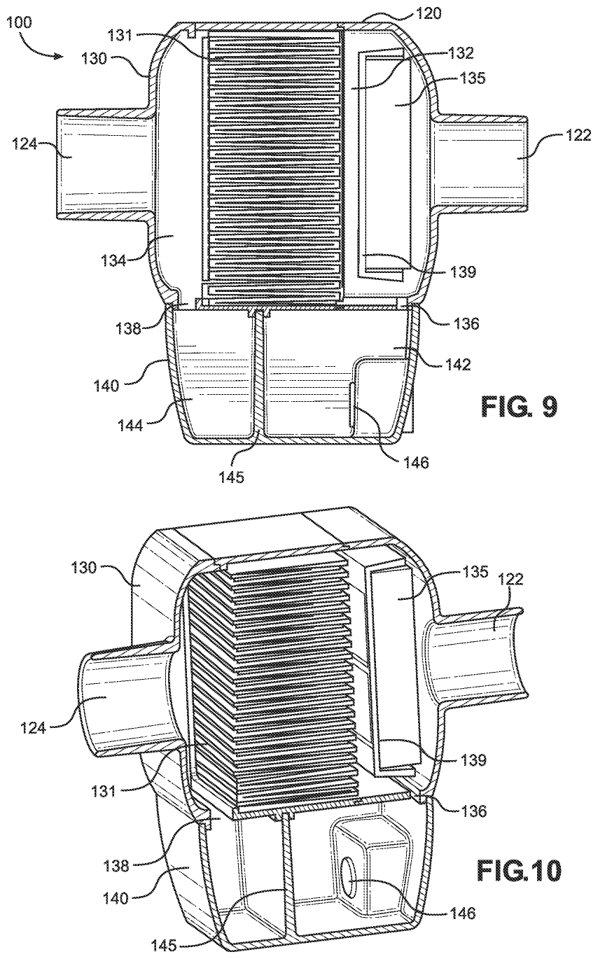

Referring to FIGS. 9 and 10, the filter member 131 is configured to fit within the filter compartment 130 such that the flow of air from the air inlet port 122 to the air outlet port 124 must pass through the filter member 131. Stated another way, the filter member 131 has a shape and size relative to a shape and size of the filter compartment 130 such that the flow of air from the air inlet port 122 to the air outlet port 124 is prevented from bypassing the filter member 131. For instance, as shown in FIGS. 9 and 10, the filter member 131 may have a generally cuboidal shape, among others.

Heated and humidified air that enters the air inlet port 122 may produce condensate within the filter compartment 130 due to the colder air within the filter compartment. Condensation or rainout may occur in the patient side 132 of the filter compartment 130 before the air flow passes through the filter member 131, and thus condensate that accumulates within the patient side of the filter compartment may be dirty water. As previously described, the filter member 131 may be capable of letting through gaseous vapor comprising water droplets. Accordingly, condensation or rainout may also occur in the ventilator side 134 of the filter compartment 130 after the air flow passes through the filter member 131, and thus condensate that accumulates within the ventilator side of the filter compartment may be clean water.

A pre-filter impact pad 135 may also be provided within the patient side 132 of the filter compartment 130. The impact pad 135 is operable to remove liquid particles from the humidified flow of air before the humidified air reaches the filter member 131. As shown in FIGS. 9 and 10, the impact pad 135 is located between the filter member 131 and the air inlet port 122. Thus, the humidified flow of air contacts the impact pad 135 before reaching the filter member 131. More particularly, the air flow is diverted around the pad and/or through the pad toward the filter member in order to enhance impaction or catching of aerosol particles.

The impact pad 135 may be absorbent and therefore able to retain the liquid particles. Further, the impact pad may be electrostatically charged for treating the flow of air from the air inlet port 122. For instance, the electrostatic impact pad may comprise a fibrous material imbued with an electrical charge during manufacturing. Thus, the electrostatic impact pad 135 is operable to attract and retain liquid particles in the air flow so as to prevent their further travel into the filter member 131, thus prolonging the lifespan of the filter member.

The impact pad 135 may have generally a planar front surface positioned so as to directly face the air inlet port 122 in a direction perpendicular to the flow of air from the air inlet port 122. Moreover, the impact pad 135 may be shaped and sized such that its cross-section is larger than a cross-section of the air inlet port 122. Thus, the impact pad 135 is located in the direct flow path of air entering through the air inlet port 122.

Further, the impact pad 135 may be mounted on a mounting frame 139 within the patient side 132 of the filter compartment 130 so that a clearance surrounding a periphery of the impact pad is formed for allowing the flow of air from the air inlet port to be diverted around the impact pad toward the filter member. The clearance defines a space or gap between the impact pad and an interior surface of the filter compartment. The clearance allows the flow of air to be diverted around a top of the pad, a bottom of the pad, and lateral sides of the pad. The impact pad 135 may be secured to the mounting frame 139 by a fastener, such as an adhesive or a clip, among others. According to other aspects, the impact pad may have a conical shape or a cylindrical shape, among others. The mounting frame 139 may also include at least one opening configured to allow the air flow to pass from the air inlet port 122 to the filter member 131 through the impact pad 135.

During use, the electrostatic impact pad 135 causes the air to diffuse, slow down, and be diverted around the pad and/or flow through the pad toward the filter member. The impact pad therefore enhances impaction or catching of aerosol particles. The electrostatic charge on the impact pad 135 attracts liquid particles from the flow of air, thereby preventing the liquid particles from travelling through the filter member 131, and also preventing the liquid particles from being diverted around the filter member. Accordingly, the electrostatic impact pad is operable to scrub out aerosol from the flow of air from the air inlet port.

The condensate collection compartment 140 is located directly adjacent to and below the filter compartment 130, and is configured to collect liquid water formed by condensation within the filter compartment. More particularly, the condensate collection compartment 140 includes a first reservoir 142 operable to collect liquid formed by condensation within the patient side 132 of the filter compartment 130, and a second reservoir 144 operable to collect liquid formed by condensation within the ventilator side 132 of the filter compartment. A partition 145 is provided within the condensate collection compartment 140 between the first and second reservoirs 142, 144 to separate the reservoirs and ensure the dirty water within the first reservoir 142 does not mix with the clean water in the second reservoir 144. In some aspects, the filter compartment 130 and the condensate collection compartment 140 may be integrally formed. In other aspects, the condensate collection compartment 140 may be removably attached to the filter compartment 130.

During use in patient respiration, liquid condensation that forms within the filter compartment 130 will drip downward toward the condensate collection compartment 140 due to the effect of gravity. More specifically, condensate within the patient side 132 of the filter compartment 130 will pass downward through a first condensate passage 136 and into the first reservoir 142 of the condensate collection compartment 140 due to gravity. Similarly, condensate within the ventilator side 134 of the filter compartment 130 will pass downward through a second condensate passage 138 and into the second reservoir 144 of the condensate collection compartment 140 due to gravity.

As also shown in FIGS. 9 and 10, a drainage port 146 is provided in a wall of the condensate collection compartment 140. The drainage port 146 may be self-sealing to prevent a leakage of air for maintaining pressure in the breathing circuit, and also to prevent a leakage of liquid. The drainage port 146 may comprise a self-closing valve assembly 50, as previously described above, where the valve assembly is operable to allow removal of the liquid collected in the first reservoir 142. In particular, the valve assembly 50 is operable to allow suctioning of the liquid condensation collected in the first reservoir 142. Such suctioning may be performed by placing a suction wand, or other suction conduit connected to a vacuum source, into the valve assembly 50. The valve assembly 50 comprises a one-way check valve 51, such as a ball check valve, and a valve cover 52 placed over the check valve. The one-way check valve 51 is configured to prevent a leakage of air in order to maintain pressure in the breathing circuit. The one-way check valve 51 also allows the liquid to flow out of the reservoir and prevents the liquid from flowing back into the reservoir.

As depicted in FIGS. 9 and 10, the drainage port 146 is provided only in a side wall of the condensate collection compartment 140 for accessing the first reservoir 142 containing dirty water. No drainage port is shown provided in a side wall of the condensate collection compartment 140 for accessing the second reservoir 144 containing clean water so as to prevent a user from using the same suction wand on both the dirty and the clean sides of the apparatus. Thus, the possibility of cross-contamination of condensate between the first and second reservoirs is eliminated or minimized. It should be appreciated, however, that a second drainage port may be provided in a wall of the condensate collection compartment 140 for allowing removal of the clean liquid collected in the second reservoir 144. Such a second drainage port may be similar to the second drainage port 48 previously discussed above.

Further, in implementations of the condensate collection compartment 140 having both first and second drainage ports, each port may be provided on different side walls of the condensate collection compartment, or on the same side wall of the condensate collection compartment to provide enhanced ergonomics. Additionally, the condensate collection compartment 140 may include a cut-out portion or stepped region configured to receive the valve assembly 50 so that it does not extend beyond an outer wall of the housing. Such an arrangement helps prevent or minimize damage to the valve assembly 50 caused by accidentally bumping it or knocking it during use.

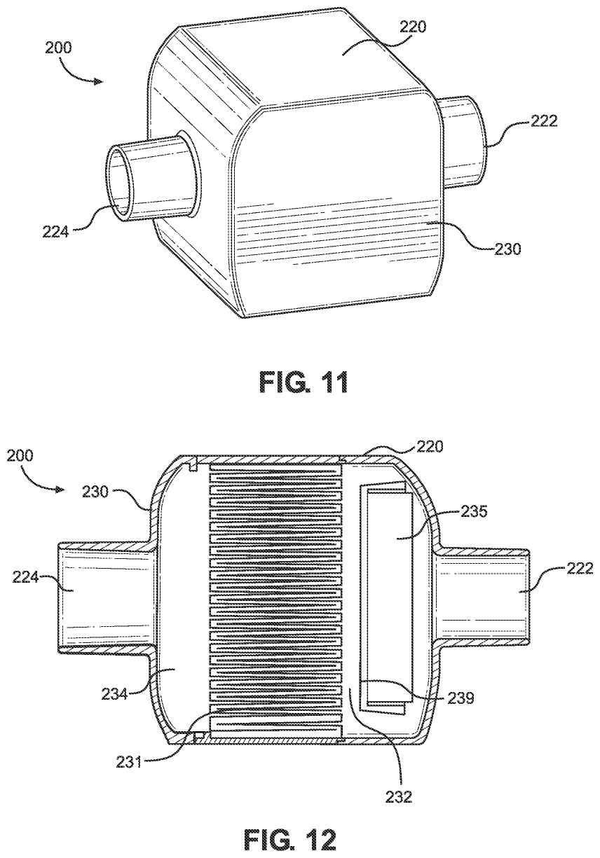

A respiratory filter and condensate management apparatus 200 for a breathing circuit is illustrated in FIGS. 11 and 12 in accordance with another implementation of the present disclosure. The respiratory filter and condensate management apparatus 200 comprises a housing 220 having a filter compartment 230. The filter compartment 230 includes an air inlet port 222 and an air outlet port 224. The air inlet port 222 is configured to connect to an expiratory limb of the breathing circuit, and the air outlet port 224 is configured to connect to a ventilator.

A filter member 231 is provided within the filter compartment 230 and is in communication with the air inlet port 222 and the air outlet port 224 for filtering bacteria, viruses, medicament, and/or other waste material exhaled by a patient during respiration. As shown, the filter member 231 is located in an air flow path from the air inlet port 222 to the air outlet port 224. Similar to the filter members previously described above, the filter member 231 may be made from a material, such as micro-fiberglass, that is operable to catch bacteria, viruses, medicament, and/or other waste material, while still allowing the flow of air to pass therethrough. For instance, the filter member 231 may be a high efficiency particulate air (HEPA) filter, or an ultra low particulate air (ULPA) filter, among others. The filter member 231 may also be pleated to improve efficiency of the air filtration. According to some aspects, the filter member 231 may also be capable of letting through gaseous vapor comprising small water droplets. Further, the filter member may be treated with an antimicrobial agent.

As shown in FIG. 12, the filter member 231 is configured to fit within the filter compartment 230 such that the flow of air from the air inlet port 222 to the air outlet port 224 must pass through the filter member 231. The filter member 231 is therefore shaped and sized relative to the filter compartment 230 to prevent the flow of air from the air inlet port 222 to the air outlet port 224 from bypassing the filter member 231. For instance, the filter member 231 may have a generally cuboidal shape, among others.

The filter compartment 230 comprises a patient side 232 adjacent to the air inlet port 222, and a ventilator side 234 adjacent to the air outlet port 224. A pre-filter impact pad 235 may be provided within the patient side 232 of the filter compartment 230 for scrubbing aerosol from the flow of air before it reaches the filter member 231. Stated another way, the impact pad 235 is operable to remove liquid particles from the humidified flow of air before the humidified air reaches the filter member 231. The impact pad 235 is located between the filter member 231 and the air inlet port 222 so that the humidified flow of air contacts the impact pad 235 before reaching the filter member 231. More particularly, the air flow may be diverted around the pad and/or through the pad toward the filter member in order to enhance impaction or catching of aerosol particles.

Additionally, the impact pad 235 may be absorbent and therefore able to retain the liquid particles. Further, the impact pad 235 may be electrostatically charged for treating the flow of air from the air inlet port 222. For instance, the electrostatic impact pad 235 may comprise a fibrous material imbued with an electrical charge during manufacturing. Thus, the electrostatic impact pad 235 is operable to attract and retain liquid particles in the air flow so as to prevent their further travel into the filter member 231, thus prolonging the lifespan of the filter member.

The impact pad 235 may have a generally planar front surface positioned so as to directly face the air inlet port 222 in a direction perpendicular to the flow of air from the air inlet port 222, as depicted in FIG. 12. Moreover, the impact pad 235 may be shaped and sized such that its cross-section is larger than a cross-section of the air inlet port 222. Thus, the impact pad 235 is located in the direct flow path of air entering through the air inlet port 222.

Further, the impact pad 235 may be mounted on a mounting frame 239 within the patient side 232 of the filter compartment 230 so that a clearance surrounding a periphery of the impact pad is formed for allowing the flow of air from the air inlet port to be diverted around the impact pad toward the filter member. The clearance defines a space or gap between the impact pad and an interior surface of the filter compartment. The clearance allows the flow of air to be diverted around a top of the pad, a bottom of the pad, and lateral sides of the pad. The impact pad 235 may be secured to the mounting frame 239 by a fastener, such as an adhesive or a clip, among others. According to other aspects, the impact pad may have a conical shape or a cylindrical shape, among others. The mounting frame 239 may also include at least one opening configured to allow the air flow to pass from the air inlet port 222 to the filter member 231 through the impact pad 235.

During use, the electrostatic impact pad 235 causes the air to diffuse, slow down, and be diverted around the pad and/or flow through the pad toward the filter member. The impact pad therefore enhances impaction or catching of aerosol particles. The electrostatic charge on the impact pad 235 attracts liquid particles from the flow of air, thereby preventing the liquid particles from travelling through the filter member 231, and also preventing the liquid particles from being diverted around the filter member. Accordingly, the electrostatic impact pad is operable to scrub out aerosol from the flow of air from the air inlet port.

While the respiratory filter and condensate management apparatus has been described in terms of what may be considered to be specific aspects, the present disclosure is not limited to the disclosed aspects. Additional modifications and improvements to the respiratory filter and condensate management apparatus may be apparent to those skilled in the art. Moreover, the many features and advantages of the disclosure are apparent from the detailed specification, and thus, it is intended by the appended claims to cover all such features and advantages of the disclosure which fall within the spirit and scope of the disclosure.

Further, it is not desired to limit the disclosure to the exact construction and operation illustrated and described, and accordingly, all suitable modifications and equivalents may be resorted to, falling within the scope of the disclosure. The present disclosure should therefore be considered as illustrative and not restrictive. As such, this disclosure is intended to cover various modifications and similar arrangements included within the spirit and scope of the claims, which should be accorded their broadest interpretation so as to encompass all such modifications and similar structures.

What is claimed is:

1. A respiratory filter and condensate management apparatus for a breathing circuit, the apparatus comprising:
a housing having an air inlet port and an air outlet port, the air inlet port configured to connect to an expiratory limb of the breathing circuit for receiving a flow of expiratory air, and the air outlet port configured to connect to a ventilator for outputting the flow of expiratory air;
a filter compartment provided within the housing, the filter compartment being in communication with the air inlet port and the air outlet port;
a filter member provided within the filter compartment and located in a flow path of the expiratory air from the air inlet port to the air outlet port;
a condensate collection compartment provided within the housing, the condensate collection compartment being adjacent to the filter compartment and including a first reservoir operable to collect liquid formed by condensation in the flow of expiratory air; and
a first self-sealing drainage port provided in a wall of the condensate collection compartment, the first self-sealing drainage port operable to allow removal of the collected liquid from the first reservoir without disconnecting the filter from the breathing circuit.

2. The respiratory filter and condensate management apparatus according to claim 1, wherein the first reservoir is configured to collect liquid within a patient side of the filter compartment before the flow of expiratory air passes through the filter member.

3. The respiratory filter and condensate management apparatus according to claim 1, further comprising a first condensate passage operable to permit liquid condensation within the filter compartment to pass into the first reservoir of the condensate collection compartment.

4. The respiratory filter and condensate management apparatus according to claim 1, wherein the first self-sealing drainage port comprises a self-closing one-way valve operable to allow suctioning out liquid collected in the first reservoir.

5. The respiratory filter and condensate management apparatus according to claim 1, wherein the condensate collection compartment further includes a second reservoir operable to collect liquid formed by condensation in the flow of expiratory air within the filter compartment.

6. The respiratory filter and condensate management apparatus according to claim 5, wherein the second reservoir is configured to collect liquid within a ventilator side of the filter compartment after the flow expiratory air passes through the filter member.

7. The respiratory filter and condensate management apparatus according to claim 5, further comprising a second self-sealing drainage port provided in a wall of the condensate collection compartment, the second self-sealing drainage port operable to allow removal of the collected liquid from the second reservoir.

8. The respiratory filter and condensate management apparatus according to claim 7, wherein the second self-sealing drainage port comprises a self-closing valve operable to allow suctioning out liquid collected in the second reservoir.

9. The respiratory filter and condensate management apparatus according to claim 5, further comprising a second condensate passage operable to permit liquid within the filter compartment to pass into the second reservoir of the condensate collection compartment.

10. The respiratory filter and condensate management apparatus according to claim 5, wherein the first and second reservoirs in the condensate collection compartment are separated by a partition configured to prevent mixing the liquid collected in the first reservoir with the liquid collected in the second reservoir.

11. The respiratory filter and condensate management apparatus according to claim 1, further comprising an impact pad within the filter compartment and operable to remove liquid from the flow of expiratory air.

12. The respiratory filter and condensate management apparatus according to claim 11, wherein the impact pad is located in a patient side of the filter compartment between the filter member and the air inlet port.

13. A respiratory filter and condensate management apparatus for a breathing circuit, the apparatus comprising:
a housing having an air inlet port and an air outlet port, the air inlet port configured to connect to a breathing circuit for receiving a flow of respiratory air, and the air outlet port configured to connect to a ventilator for outputting the flow of respiratory air;
a filter compartment provided within the housing, the filter compartment defining a patient side in communication with the air inlet port and a ventilator side in communication with the air outlet port;
a filter member provided within the filter compartment and located in a flow path of the respiratory air from the air inlet port to the air outlet port;
a condensate collection compartment provided within the housing and including a reservoir and a self-sealing drainage port,
the reservoir configured to collect liquid formed by condensation in the flow of respiratory air within the filter compartment, and
the self-sealing drainage port operable to prevent a leakage of the respiratory air for maintaining pressure in the breathing circuit.

14. The respiratory filter and condensate management apparatus according to claim 13, wherein the self-sealing drainage port comprises a one-way valve operable to allow removal of the collected liquid from the reservoir while the housing is connected to the breathing circuit.

15. The respiratory filter and condensate management apparatus according to claim 13, further comprising an impact pad within the filter compartment and operable to remove liquid from the flow of respiratory air.

16. The respiratory filter and condensate management apparatus according to claim 15, wherein the impact pad is located in the patient side of the filter compartment between the filter member and the air inlet port.

17. An apparatus for removing liquid particles from a flow of respiratory air within a breathing circuit, the apparatus comprising:
a housing having an air inlet port and an air outlet port, the air inlet port configured to connect to a breathing circuit for receiving a flow of respiratory air, and the air outlet port configured to connect to a ventilator for outputting the flow of respiratory air;
a filter compartment provided within the housing, the filter compartment defining a patient side in communication with the air inlet port and a ventilator side in communication with the air outlet port;
a filter member provided within the filter compartment and located in a flow path of the respiratory air from the air inlet port to the air outlet port; and
an impact pad provided within the patient side of the filter compartment and operable to remove liquid particles from the flow of respiratory air.

18. The apparatus according to claim 17, further comprising a condensate collection compartment provided within the housing and including a reservoir and a self-sealing drainage port.

19. The apparatus according to claim 18, wherein the reservoir is configured to collect liquid formed by condensation in the flow of respiratory air within the filter compartment.

20. The apparatus according to claim 18, wherein the self-sealing drainage port is operable to prevent a leakage of the respiratory air for maintaining pressure in the breathing circuit.
